(19) United States
(12) Patent Application Publication
(10) Pub. No.: US 2014/0330370 A1
(43) Pub. Date: Nov. 6, 2014

(54) Prosthetic Valve Delivery and Mounting Apparatus and System

(71) Applicants: Robert G. Matheny, Norcross, GA (US); James Cox, Ladue, MO (US); Anna Fallon, Woodstock, GA (US)

(72) Inventors: Robert G. Matheny, Norcross, GA (US); James Cox, Ladue, MO (US); Anna Fallon, Woodstock, GA (US)

(21) Appl. No.: 14/264,746

(22) Filed: Apr. 29, 2014

Related U.S. Application Data
(60) Provisional application No. 61/819,275, filed on May 3, 2013.

(51) Int. Cl. A61F 2/24

(52) U.S. Cl. CPC A61F 2/2445; A61F 2/2418
USPC 623/2.14; 623/2.37

(57) ABSTRACT

A valve mounting apparatus having an elongated tubular mesh structure with proximal and distal ends, a cardiovascular structure engagement region disposed between the proximal and distal ends, and a lumen therethrough, the distal end of the structure including a valve engagement region that is configured to engage a first end of a prosthetic valve, the mesh structure being configured to transition from a pre-deployment configuration, wherein the structure is capable of being disposed at a cardiovascular valve region, such as a valve annulus region, to a post-deployment expanded configuration, wherein the cardiovascular structure engagement region is anchored to the cardiovascular valve region.

### CROSS-REFERENCE TO RELATED APPLICATIONS

This application claims the benefit of U.S. Application No. 61/819,275, filed on May 3, 2013.

### FIELD OF THE INVENTION

The present invention generally relates to prosthetic valves for replacing defective cardiovascular valves. More particularly, the present invention relates to a prosthetic valve delivery and mounting apparatus and system.

### BACKGROUND OF THE INVENTION

As is well known in the art, the human heart has four valves that control blood flow circulating through the human body. Referring to FIGS. 1A and 1B, on the left side of the heart 100 is the mitral valve 102, located between the left atrium 104 and the left ventricle 106, and the aortic valve 108, located between the left ventricle 106 and the aorta 110. Both of these valves direct oxygenated blood from the lungs into the aorta 110 for distribution through the body.

The tricuspid valve 112, located between the right atrium 114 and the right ventricle 116, and the pulmonary valve 118, located between the right ventricle 116 and the pulmonary artery 120, however, are situated on the right side of the heart 100 and direct deoxygenated blood from the body to the lungs.

There are also five papillary muscles in the heart 100; three in the right ventricle 116 and two in the left ventricle 106. The anterior, posterior and septal papillary muscles of the right ventricle 116 each attach via chordae tendinae to the tricuspid valve 112. The anterior and posterior papillary muscles of the left ventricle 106 attach via chordae tendinae to the mitral valve 102.

Since heart valves are passive structures that simply open and close in response to differential pressures, the issues that can develop with valves are typically classified into two categories: (i) stenosis, in which a valve does not open properly, and (ii) insufficiency (also called regurgitation), in which a valve does not close properly. Stenosis and insufficiency can occur concomitantly in the same valve or in different valves.

Both of the noted valve abnormalities can adversely affect organ function and result in heart failure. For example, insufficiency of the inlet (atrioventricular) tricuspid valve 112 to the right ventricle 116 of the heart 100 results in regurgitation of blood back into the right atrium 114, which, serving to receive blood flow returning in the veins from the entire body, then results in turn in suffusion and swelling (edema) of all the organs, most notably in the abdomen and extremities, insufficient forward conduction of blood flow from the right ventricle 116 into the lungs causing compromise of pulmonary function, and ultimately pump failure of the right heart. Collectively these conditions (collectively deemed right heart failure) can, and in many instances will, lead to incapacity and, possibly, death if progressive and uncorrected.

In addition to stenosis and insufficiency of a heart valve, surgical intervention may also be required for certain types of bacterial or fungal infections, wherein the valve may continue to function normally, but nevertheless harbors an overgrowth of bacteria (i.e. “vegetation”) on the valve leaflets. The vegetation can, and in many instances will, flake off (i.e. “embolize”) and lodge downstream in a vital artery.

If such vegetation is present on the valves of the left side (i.e., the systemic circulation side) of the heart, embolization can, and often will, result in sudden loss of the blood supply to the affected body organ and immediate malfunction of that organ. The organ most commonly affected by such embolization is the brain, in which case the patient can, and in many instances will, suffer a stroke.

Likewise, bacterial or fungal vegetation on the tricuspid valve can embolize to the lungs. The noted embolization can, and in many instances will, result in lung dysfunction.

Treatment of the noted heart valve dysfunctions typically comprises reparation of the diseased heart valve with preservation of the patient's own valve or replacement of the valve with a mechanical or bioprosthetic valve, i.e. a prosthetic valve.

Various prosthetic heart valves have thus been developed for replacement of natural diseased or defective heart valves. Illustrative are the tubular prosthetic tissue valves disclosed in Applicant's Co-Pending U.S. application Ser. Nos. 13/560,573, 13/782,024, 13/782,289, 13/804,683, 13/182,170, 13/480,347 and 13/480,324. A further tubular prosthetic valve is disclosed in U.S. Pat. Nos. 8,257,434 and 7,998,196.

Heart valve replacement requires a great deal of skill and concentration to achieve a secure and reliable attachment of a prosthetic valve to a vessel and/or cardiovascular structure or tissue. Various surgical methods for implanting a prosthetic valve have thus been developed.

The most common surgical method that is employed to implant a prosthetic atrioventricular valve (mitral or tricuspid) comprises suturing the proximal end of the valve to cardiovascular tissue proximate the original mitral or tricuspid valve position and the distal end (or a segment of one or more valve leaflets) directly to the papillary muscles.

There are numerous drawbacks and disadvantages associated with suturing a prosthetic valve to a cardiovascular tissue and structures; particularly, the papillary muscles. A major disadvantage is the high risk of perivalvular leakage.

Another major problem associated with suturing a valve directly to the papillary muscles is that the region proximate thereto is subject to extreme stress (induced by cardiac cycles), which can, and in most instances will, adversely affect the structural integrity of the valve.

There is thus a need to provide a prosthetic valve mounting system that overcomes the drawbacks and disadvantages associated with conventional prosthetic valve implantation methods and systems.

There is also a need to provide prosthetic atrioventricular tissue valves and methods for attaching same to cardiovascular structures and/or tissue that maintain or enhance the structural integrity of the valve when subjected to cardiac cycle induced stress.

It is therefore an object of the present invention to provide improved methods for securely attaching prosthetic atrioventricular valves to cardiovascular structures and/or tissue.

It is another object of the present invention to provide a prosthetic valve delivery and mounting system that is configured to securely and sealingly connect prosthetic atrioventricular valves to cardiovascular structures and/or tissue.

It is another object of the present invention to provide prosthetic atrioventricular tissue valves and methods for attaching same to cardiovascular structures and/or tissue that enhance the structural integrity of the valve.

It is another object of the present invention to provide reinforced prosthetic atrioventricular tissue valves and methods for attaching same to cardiovascular structures and/or tissue that preserve the structural integrity of the cardiovascular structure(s) when attached thereto.

It is another object of the present invention to provide prosthetic atrioventricular tissue valves having means for secure, reliable, and consistently highly effective attachment to cardiovascular structures and/or tissue.

It is another object of the present invention to provide prosthetic atrioventricular tissue valves that induce host tissue proliferation, bioremodeling and regeneration of new tissue and tissue structures with site-specific structural and functional properties.

It is another object of the present invention to provide prosthetic atrioventricular tissue valves that are capable of administering a pharmacological agent to host tissue and, thereby produce a desired biological and/or therapeutic effect.

### SUMMARY OF THE INVENTION

The present invention is directed to a delivery and mounting apparatus for prosthetic atrioventricular valves that can be readily employed to selectively replace diseased or defective mitral and tricuspid valves.

The present invention is also directed to prosthetic atrioventricular valves having a delivery and mounting apparatus of the invention.

As discussed in detail herein, the mounting apparatus is designed and configured to sealingly engage prosthetic atrioventricular valves and anchor the atrioventricular valves to selective cardiovascular structures.

In a preferred embodiment of the invention, the mounting apparatus comprises an elongated radially expandable and compressible mesh structure having proximal and distal ends, a cardiovascular structure engagement region disposed between the proximal and distal ends, and a lumen therethrough.

In a preferred embodiment of the invention, the proximal and distal ends of the mesh structure include a valve engagement region that is configured to engage an end, e.g., proximal end, of a prosthetic tissue valve, whereby the end of the prosthetic valve is disposed in the mesh structure lumen.

According to the invention, the cardiovascular structure engagement region is configured engage the ventricular and atrial sides of a valve annulus and anchor the mounting apparatus and, hence, prosthetic valve connected thereto, to the valve annulus. In some embodiments, the cardiovascular structure engagement region is configured to sealingly engage the ventricular and atrial sides of a valve annulus.

In some embodiments, the valve annulus is disposed proximate a mitral valve region.

In some embodiments, the valve annulus is disposed proximate a tricusped valve region.

In some embodiments, the mesh structure comprises a biocompatible polymeric material.

In some embodiments, the mesh structure comprises (Artelon®).

In some embodiments, the mesh structure further includes an impermeable liner.

In some embodiments, the liner comprises an ECM material, e.g., ECM sheet.

In a preferred embodiment of the invention, the prosthetic atrioventricular valves comprise continuous tubular members.

In some embodiments of the invention, the tubular members comprise an ECM material.

In a preferred embodiment of the invention, the ECM material comprises mammalian extracellular matrix tissue selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof.

In some embodiments of the invention, the ECM liner, if employed, and/or tubular members (or a portion thereof) include at least one additional biologically active agent or composition, i.e. an agent that induces or modulates a physiological or biological process, or cellular activity, e.g., induces proliferation, and/or growth and/or regeneration of tissue.

In some embodiments, the ECM liner, if employed, and/or tubular members (or a portion thereof) include at least one pharmacological agent or composition (or drug), i.e. an agent or composition that is capable of producing a desired biological effect in vivo, e.g., stimulation or suppression of apoptosis, stimulation or suppression of an immune response, etc.

In some embodiments, the tubular members include a reinforcement member (i.e. tubular member constructs) that is designed and configured to enhance the structural integrity of the tubular member and, hence, prosthetic atrioventricular tissue valve formed therefrom, and facilitate secure engagement of the prosthetic valve to cardiovascular structures, e.g., selective papillary muscles, ventricles, etc., and/or cardiovascular tissue.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A and 1B are schematic illustrations of a human heart, showing blood flow therethrough;

FIG. 2A is a perspective view of one embodiment of a prosthetic valve delivery and mounting apparatus in a pre-deployment (or compressed) configuration, in accordance with the invention;

FIG. 2B is a perspective view of another embodiment of a prosthetic valve delivery and mounting apparatus in a pre-deployment (or compressed) configuration, in accordance with the invention;

FIG. 3 is a perspective view of the prosthetic valve delivery and mounting apparatus shown in FIG. 2A in a post-deployment (or expanded) configuration, in accordance with the invention; and

FIGS. 4 and 5 are illustrations of the mounting apparatus shown in FIGS. 2A and 3 positioned proximate a mitral valve position within a human heart, in accordance with the invention.

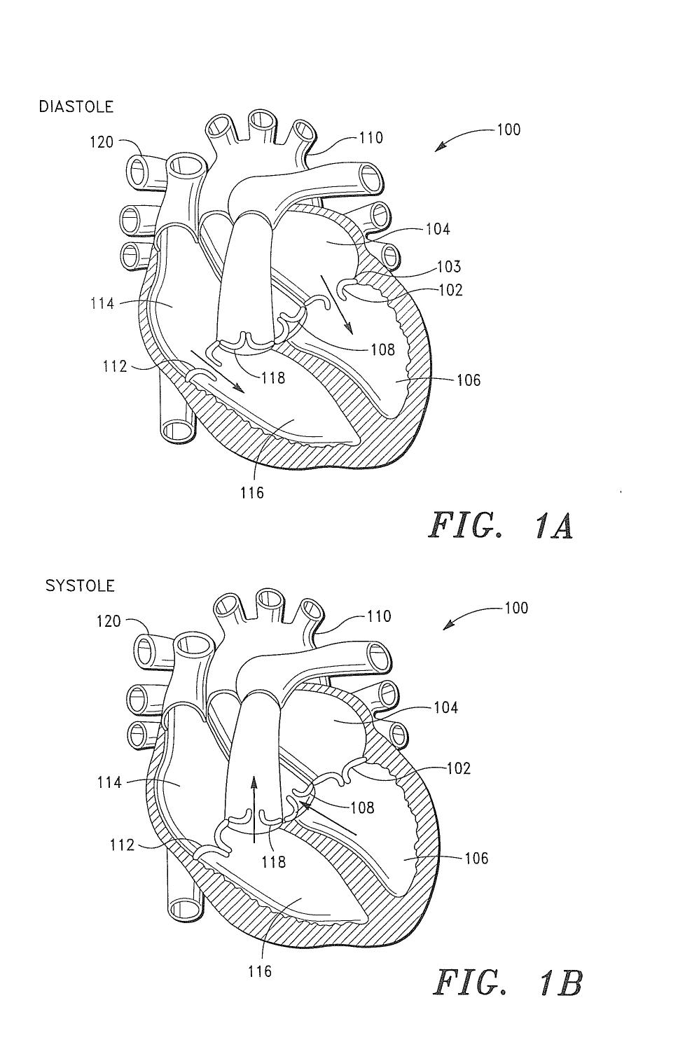

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### DEFINITIONS

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornament=extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The term “Artelon”, as used herein, means a poly(urethane urea) material distributed by Artimplant AB in Goteborg, Sweden.

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “therapeutically effective”, as used herein, means that the amount of the “pharmacological agent” and/or “biologically active agent” and/or “pharmacological composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As stated above, the present invention is directed to delivery and mounting apparatus for prosthetic atrioventricular valves that can be readily employed to selectively replace diseased or defective mitral and tricuspid valves.

The present invention is also directed to prosthetic atrioventricular valves having a delivery and mounting apparatus of the invention.

As discussed in detail herein, the mounting apparatus is designed and configured to engage prosthetic atrioventricular valves and anchor the atrioventricular valves to selective cardiovascular structures.

According to the invention, the mounting apparatus (and prosthetic valves attached thereto) can be delivered to a desired cardiovascular position or structure via conventional open heart surgery and/or via conventional percutaneous (or transcatheter) delivery techniques.

In a preferred embodiment of the invention, the mounting apparatus comprises an elongated radially expandable and compressible mesh structure having proximal and distal ends, valve engagement regions disposed proximate the proximal and distal ends, a cardiovascular structure engagement region disposed between the valve engagement regions, and a lumen therethrough.

According to the invention, the cardiovascular structure engagement region is configured engage a selective cardiovascular structure; particularly, the ventricular and atrial sides of a valve annulus, and anchor the mounting apparatus (and, hence, prosthetic valve associated therewith) thereto. In some embodiments, the cardiovascular structure engagement region is configured to sealingly engage the ventricular and atrial sides of a valve annulus.

In some embodiments, the valve annulus comprises a mitral valve annulus.

In some embodiments, the valve annulus comprises a tricusped valve annulus.

In some embodiments, the valve engagement region comprises a valve engagement member.

In a preferred embodiment, the mesh structure has a pre-deployment length that extends from the proximal to distal ends in the range of approximately 3 mm-5 cm.

In some embodiments, the pre-deployment length is in the range of approximately 3 mm-1 cm.

In some embodiments, the pre-deployment length is in the range of approximately 5 mm-10 mm.

In a preferred embodiment, the mesh structure has a pre-deployment outer diameter in the range of approximately 8 mm-6 cm.

In some embodiments, the mesh structure comprises a biocompatible polymeric material.

In some embodiments, the polymeric material comprises Artelon®.

In some embodiment of the invention, the mesh structure comprises a biocompatible metal, more preferably, a biocompatible and biodegradeable metal. Suitable metals thus include, without limitation, stainless steel, magnesium and Nitinol®.

In some embodiments of the invention, the mounting apparatus or mesh structure further includes an impermeable liner.

According to the invention, the liner can comprise various natural and synthetic biocompatible materials, including, without limitation, collagen, Dacron®, and like materials.

In a preferred embodiment, the liner comprises a first extracellular matrix (ECM) material.

In some embodiments, the liner comprises an ECM sheet.

In a preferred embodiment, the ECM sheet is disposed in the mesh structure lumen proximate the lumen/mesh structure interior surface.

In a preferred embodiment, the prosthetic atrioventricular valves of the invention comprise continuous tubular members, such as the tubular prosthetic valves disclosed in Co-Pending U.S. application Ser. Nos. 13/480,347 and 13/480,324; which are incorporated by reference herein.

In some embodiments of the invention, the tubular members comprise a second ECM material.

According to the invention, the first and second ECM materials can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety.

In a preferred embodiment, the mammalian tissue sources include, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

In a preferred embodiment, the mammalian tissue source comprises an adolescent tissue source, i.e. a tissue source less than three (3) years of age.

In some embodiments, the mammalian tissue source comprises SIS.

In some embodiments, the mammalian tissue source comprises mesothelial tissue.

In some embodiments of the invention, the mesh structure liner and/or tubular member (or a portion thereof) includes at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells and proteins.

In some embodiments, the mesh structure and/or mesh structure liner and/or tubular member (or a portion thereof) includes at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises an anti-inflammatory agent.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

In some embodiments of the invention, the pharmacological agent comprises chitosan.

In some embodiments, the tubular valve members include a cardiovascular structure engagement means that is designed and configured to facilitate secure engagement of the distal end of the tubular member and, hence, prosthetic atrioventricular tissue valve formed therefrom to cardiovascular structures, e.g., selective papillary muscles, ventricles, etc., and enhance the structural integrity of the prosthetic valve. Suitable reinforcement members are disclosed in Co-Pending U.S. application Ser. No. 14/229,854, which is incorporated by reference herein.

In some embodiments, the tubular valve member include a reinforcement member that enhances the structural integrity of the tubular member and, hence, prosthetic atrioventricular tissue valve formed therefrom. Suitable reinforcement members are disclosed in Co-Pending U.S. application Ser. No. 14/264,308, filed on Apr. 29, 2014, which is incorporated by reference herein in its entirety.

In some embodiments of the invention, the reinforcement member includes anchoring means that is designed and configured to position the prosthetic valves proximate a cardiovascular structure or tissue, and maintain contact therewith for a pre-determined period of time. Suitable anchoring means are disclosed in Co-pending U.S. application Ser. Nos. 13/686,131, 13/782,024 and 13/804,683; which are incorporated by reference herein in their entirety.

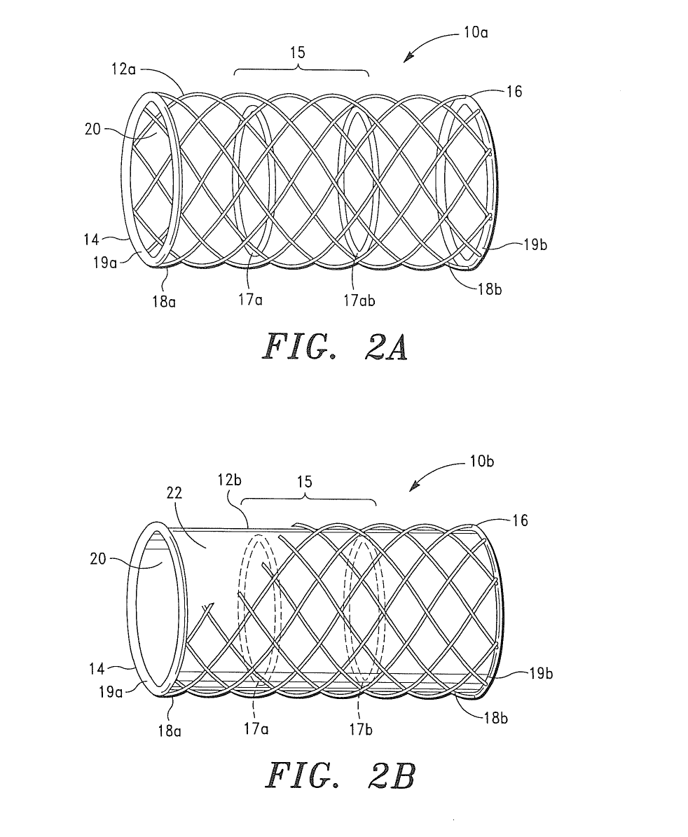

Referring now to FIGS. 2A and 3, one embodiment of a prosthetic valve mounting apparatus will be described in detail.

As illustrated in FIGS. 2A and 3, the mounting apparatus 10a comprises an elongated mesh structure 12a having proximal and distal ends 14, 16, and a central lumen 20 that allows blood flow therethrough, as denoted by arrow B_{f}. In a preferred embodiment, the mesh structure 12a is also radially expandable and compressible.

As further illustrated in FIGS. 2 and 3, the mounting apparatus 10a further includes a cardiovascular structure engagement region 15 that is disposed between the proximal and distal ends 14, 16. In a preferred embodiment, the cardiovascular engagement region 15 includes spaced reinforcement rings 17a, 17b, which, in some embodiments, define the cardiovascular engagement region 15. According to the invention, the reinforcement rings 17a, 17b can also be disposed within the cardiovascular engagement region 15.

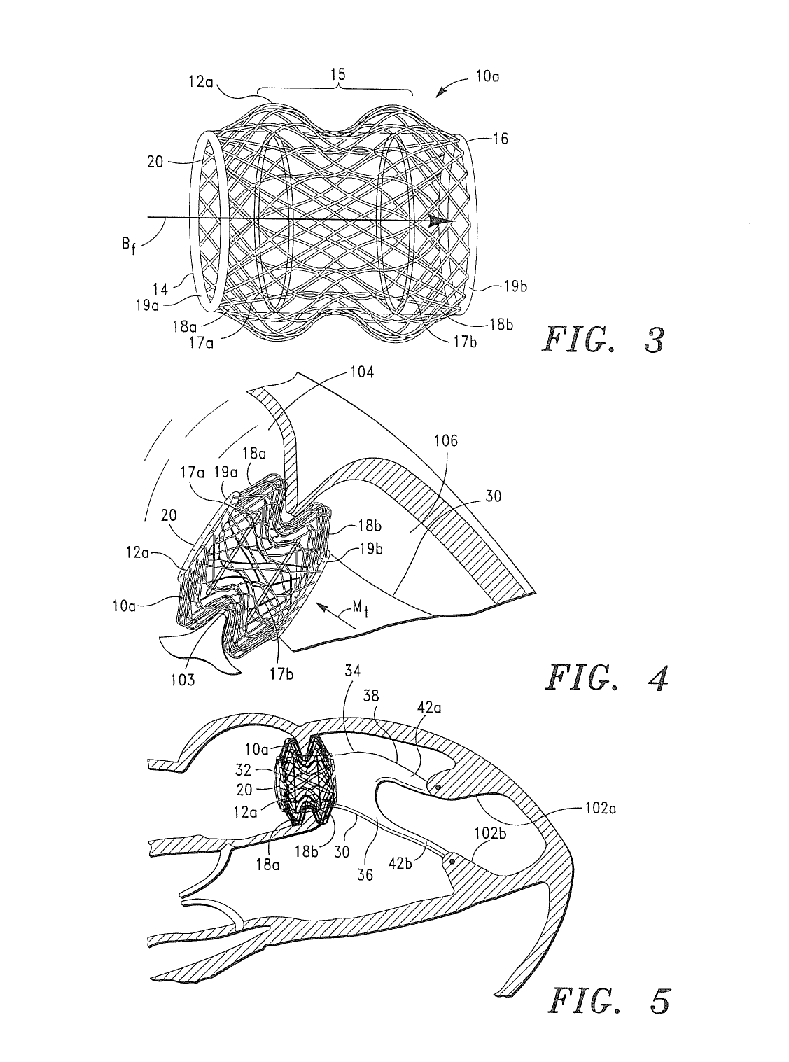

Referring now to FIGS. 4 and 5, the proximal and distal ends 14, 16 of the mesh structure 12a include valve engagement regions 18a, 18b that are configured to engage an end, preferably, a proximal end 32, of a prosthetic tissue valve 30, whereby the prosthetic tissue valve 30 is disposed in the mesh structure lumen 20.

In the illustrated embodiment, the valve engagement regions 18a, 18b include valve engagement members 19a, 19b that are configured to facilitate engagement of the mounting apparatus 10a to the proximal end 32 of the valve 30. According to the invention, the valve engagement members 19a, 19b can similarly comprise a biocompatible polymeric material or a different material, such as, by way of example, stainless steel, magnesium, Nitinol®.

As discussed in detail below, the valve engagement members 19a, 19b can be secured to the proximal end 32 of the valve 30 by various conventional means. In a preferred embodiment, the engagement members 19a, 19b are sutured to the proximal end 32 of the valve 30.

In a preferred embodiment of the invention, the mesh structure 12a and, thereby, mounting apparatus 10a is capable of transitioning from a pre-deployment configuration (shown in FIG. 2A), wherein the mounting apparatus 10a (and attached prosthetic valve, e.g., valve 30) are capable of being disposed at a mitral or tricuspid valve region and/or a desired cardiovascular structure region, to a post-deployment compressed configuration (shown in FIG. 3), wherein the cardiovascular structure engagement region 15 is disposed proximate cardiovascular tissue surrounding a mitral or tricuspid valve region, e.g., the ventricular and atrial sides of the valve annulus, or target cardiovascular structure.

According to the invention, the mesh structure 12a (and 12b, discussed below) can comprise various biocompatible materials. Thus, in some embodiments, the mesh structure 12a comprises a polymeric material, more preferably, a biocompatible polymeric material.

In some embodiments, the mesh structure 12a comprises Dacron®.

In a preferred embodiment, the mesh structure 12a comprises Artelon®.

In some embodiments of the invention, the mesh structure 12a (and 12b) includes at least one of the aforementioned biologically active or pharmacological agents or compositions, incorporated therein or coated thereon.

Referring now to FIG. 2B, there is shown another embodiment of a mesh structure 12b. As illustrated in FIG. 2b, in this embodiment, the mesh structure 12b includes a substantially impermeable liner 22.

In some embodiments of the invention, the mesh structure liner 22 comprises an extracellular matrix (ECM) material layer.

As illustrated in FIG. 2B, the ECM layer 22 is preferably disposed in the mesh structure lumen 20 proximate the surface thereof.

According to the invention, the ECM layer 22 can also be disposed on the outer surface of the mesh structure 12.

In some embodiments of the invention, at least a portion of the mesh structure 12a (and/or 12b) includes an ECM coating.

According to the invention, the ECM referenced above can be derived from various mammalian tissue sources including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

In some embodiments of the invention, the ECM inner and/or outer layer includes at least one of the aforementioned biologically active or pharmacological agents or compositions.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

As indicated above, the mounting apparatus 10a is configured to be mounted proximate selective cardiovascular structures; particularly, mitral and tricuspid valve regions.

Referring to FIG. 4, wherein the mounting apparatus 10a is mounted proximate a mitral valve region, the mounting apparatus 10a is preferably configured to engage the ventricular and atrial sides of the mitral valve annulus 103. If mounting apparatus 10b is employed, the mounting apparatus 10b is preferably configured to sealably engage the ventricular and atrial sides of the mitral valve annulus 103.

As also illustrated in FIGS. 4 and 5 and discussed above, the valve engagement regions 18a, 18b of the mounting apparatus 10a are also preferably configured to engage the proximal end 32 of the prosthetic atrioventricular valve 30.

In operation, the prosthetic tissue valve 30 is initially positioned in the mesh structure lumen 20. Valve engagement member 19a is then secured to the proximal end 32 of the valve 30 (at a forward position proximate the opening of the valve lumen) with the mounting apparatus 10a in the pre-deployment configuration shown in FIG. 2A.

After the mounting apparatus 10a is positioned in the valve annulus 103, the mounting apparatus 10a is placed in a post-deployment configuration (as shown if FIGS. 4 and 5), wherein the cardiovascular structure engagement region 15 is disposed adjacent cardiovascular tissue 105 of the valve annulus 103. In some embodiments of the invention, the mounting apparatus 10a is placed in the illustrated post-deployment configuration by moving valve engagement region 18b and, hence, valve engagement member 19b forward in the direction denoted by Arrow M_{T} wherein the mesh structure 12a is compressed. The valve engagement member 19b is then also secured to the proximal end 32 of the valve 30.

As indicated above, the valve engagement members 19a, 19b can be secured to the proximal end 32 of the valve 30 by various conventional means. In a preferred embodiment, the valve engagement members 19a, 19b are sutured to the proximal end 32 of the valve 30.

In the illustrated embodiment, the prosthetic atrioventricular valve 30 comprises a continuous tubular member 34. In some embodiments of the invention, the tubular member 34 and, hence, prosthetic valve 30 formed therefrom includes at least one internal leaflet, such as disclosed in Co-Pending U.S. application Ser. Nos. 13/804,683 and 13/782,289.

In some embodiments, the tubular member 34 includes a leaflet forming interior surface, such as disclosed in Co-Pending U.S. application Ser. No. 13/480,324 and 13/480,347.

According to the invention, the tubular member 34 (and, hence, valve 30) can also comprise various biocompatible materials.

In some embodiments of the invention, the tubular member 34 comprises a biocompatible polymeric or synthetic material.

In a preferred embodiment, the tubular member 34 and, hence, prosthetic tissue valve 30 formed therefrom comprises an ECM material.

According to the invention, the ECM material can be derived from any of the aforementioned mammalian tissue sources including, without limitation, small intestine submucosa (SIS), urinary bladder submucosa (UBS), and epithelium of mesodermal origin, i.e. mesothelial tissue.

In some embodiments of the invention, the tubular member 34 includes at least one of the aforementioned biologically active and/or pharmacological agents or compositions.

As illustrated in FIG. 4, the distal end 36 of the tubular member 34 further includes cardiovascular structure engagement means 38 that is designed and configured to securely engage the member 34 and, hence, prosthetic atrioventricular tissue valve 30 formed therefrom to cardiovascular structures, such as selective papillary muscles 102a, 102b, and/or cardiovascular tissue.

As indicated above, suitable cardiovascular structure engagement means 38 are disclosed in Co-Pending U.S. application Ser. No. 14/229,854.

In the illustrated embodiment, the cardiovascular structure engagement means 38 comprises a pair of cardiovascular structure engagement members 42a, 42b, which extend from the tubular member 12 to mimic the chordae tendinae.

As also indicated above, the tubular member 34 can also include a reinforcement member that is designed and configured to enhance the structural integrity of the tubular member 34 and, hence, prosthetic atrioventricular tissue valve 30 formed therefrom.

In some embodiments of the invention, the reinforcement member includes anchoring means that is designed and configured to position the prosthetic valve 30 proximate a cardiovascular structure or tissue, and maintain contact therewith for a pre-determined period of time. As indicated above, suitable anchoring means are disclosed in Co-pending U.S. application Ser. Nos. 13/686,131, 13/782,024 and 13/804,683; which are incorporated by reference herein in their entirety.

As will readily be appreciated by one having ordinary skill in the art, the present invention provides numerous advantages compared to prior art methods for implanting prosthetic valves. Among the advantages are the following:
The provision of improved methods for securely attaching prosthetic atrioventricular valves to cardiovascular structures and/or tissue.
The provision of a prosthetic valve delivery and mounting system that is configured to securely and sealingly engage prosthetic atrioventricular valves to cardiovascular structures and/or tissue.
The provision of prosthetic atrioventricular tissue valves and methods for attaching same to cardiovascular structures and/or tissue, which enhance the structural integrity of the valve.
The provision of prosthetic atrioventricular tissue valves and methods for attaching same to cardiovascular structures and/or tissue, which preserve the structural integrity of the cardiovascular structure(s) when attached thereto.
The provision of prosthetic atrioventricular tissue valves having means for secure, reliable, and consistently highly effective attachment to cardiovascular structures and/or tissue.
The provision of prosthetic atrioventricular tissue valves that induce host tissue proliferation, bioremodeling and regeneration of new tissue and tissue structures with site-specific structural and functional properties.
The provision of prosthetic atrioventricular tissue valves that are capable of administering a pharmacological agent to host tissue and, thereby produce a desired biological and/or therapeutic effect.
The provision of prosthetic valve mounting structures and prosthetic atrioventricular tissue valves that exhibit optimum mechanical compatibility with cardiovascular structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A valve mounting apparatus, comprising:
an elongated tubular mesh structure having proximal and distal ends and a lumen therethrough, said mesh structure being configured to receive a proximal end of a prosthetic valve therein,
said proximal end of said mesh structure including a first valve engagement region that is configured to engage a first region of a first end of said prosthetic valve, said distal end of said mesh structure including a second valve engagement region that is configured to engage a second region of said first end of said prosthetic valve,
said mesh structure further including a cardiovascular structure engagement region disposed between said mesh structure proximal and distal ends,
said mesh structure being configured to transition from a pre-deployment configuration, wherein said mesh structure is capable of being disposed at a first cardiovascular valve region, to a post-deployment compressed configuration, wherein said cardiovascular structure engagement region is disposed adjacent cardiovascular tissue disposed proximate said first cardiovascular valve region.

2. The valve mounting apparatus of claim 1, wherein said first cardiovascular valve region comprises a valve annulus having ventricular and atrial regions, and wherein, when said mesh structure is in said post-deployment configuration, said cardiovascular structure engagement region is anchored to said ventricular and atrial regions.

3. The valve mounting apparatus of claim 2, wherein said first cardiovascular valve region comprises a mitral valve region.

4. The valve mounting apparatus of claim 2, wherein said first cardiovascular valve region comprises a tricusped valve region.

5. The valve mounting apparatus of claim 1, wherein said mesh structure has a pre-deployment length extending from said mesh structure proximal end to said mesh structure distal end in the range of 3 mm-5 cm.

6. The valve mounting apparatus of claim 1, wherein said mesh structure has a pre-deployment diameter in the range of 8 mm-6 cm.

7. The valve mounting apparatus of claim 1, wherein said mesh structure comprises a biocompatible polymeric material.

8. The valve mounting apparatus of claim 7, wherein said polymeric material comprises a polymeric material selected from the group consisting of Dacron® and Artelon®.

9. The valve mounting apparatus of claim 7, wherein said polymeric material further includes first biologically active agent.

10. The valve mounting apparatus of claim 9, wherein said first biologically active agent is dispersed in said polymeric material.

11. The valve mounting apparatus of claim 9, wherein said first biologically active agent comprises an active agent coating.

12. The valve mounting apparatus of claim 9, wherein said first biologically active agent comprises a growth factor selected from the group consisting of a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

13. The valve mounting apparatus of claim 9, wherein said first biologically active agent comprises a cell selected from the group consisting of a human embryonic stem cell, fetal cardiomyocyte, myofibroblast, mesenchymal stem cell, autotransplated expanded cardiomyocyte, adipocyte, totipotent cell, pluripotent cell, blood stem cell, myoblast, adult stem cell, bone marrow cell, parenchymal cell, epithelial cell, endothelial cell, mesothelial cell, fibroblast, osteoblast, chondrocyte, exogenous cell, endogenous cell, hematopoietic stem cell, myocardial cell, skeletal cell, multi-potent progenitor cell, unipotent progenitor cell, macrophage, xenogenic cell and allogenic cell.

14. The valve mounting apparatus of claim 7, wherein said polymeric material further comprises a pharmacological agent selected from the group consisting of antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

15. The valve mounting apparatus of claim 1, wherein said mesh structure further comprises an impermeable liner, said liner being disposed in said mesh structure lumen.

16. The valve mounting apparatus of claim 15, wherein said liner comprises a first extracellular matrix (ECM) material.

17. The valve mounting apparatus of claim 16, wherein said first ECM material is derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

18. The valve mounting apparatus of claim 17, wherein said first ECM material further comprises an exogenously added second biologically active agent.

19. The valve mounting apparatus of claim 18, wherein said second biologically active agent comprises a growth factor selected from the group consisting of a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

20. The valve mounting apparatus of claim 18, wherein said second biologically active agent comprises a cell selected from the group consisting of a human embryonic stem cell, fetal cardiomyocyte, myofibroblast, mesenchymal stem cell, autotransplated expanded cardiomyocyte, adipocyte, totipotent cell, pluripotent cell, blood stem cell, myoblast, adult stem cell, bone marrow cell, parenchymal cell, epithelial cell, endothelial cell, mesothelial cell, fibroblast, osteoblast, chondrocyte, exogenous cell, endogenous cell, hematopoietic stem cell, myocardial cell, skeletal cell, multi-potent progenitor cell, unipotent progenitor cell, macrophage, xenogenic cell and allogenic cell.

21. The valve mounting apparatus of claim 16, wherein said first ECM material further comprises a pharmacological agent selected from the group consisting of antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

22. A prosthetic atrioventricular tissue valve, comprising:
a continuous tubular member formed from a first biocompatible material, said tubular member having first proximal and distal ends and a first lumen therethrough, and at least one valve leaflet formed therein,
said first distal end of said tubular member including cardiovascular structure engagement means for connecting said tubular member to a cardiovascular structure, said cardiovascular structure engagement means comprising first and second elongated members that extend distally from said first distal end of said tubular member,
said first elongated member comprising a looped member having first and second ends, said first end of said first elongated member being attached to a first point on said first distal end of said tubular member and said second end of said first elongated member being attached to a second point on said first distal end of said tubular member,
said second elongated member comprising a looped member having third and fourth ends, said third end of said second elongated member being attached to a third point on said first distal end of said tubular member and said fourth end of said second elongated member being attached to a fourth point on said first distal end of said tubular member; and
a valve mounting apparatus comprising an elongated tubular mesh structure having proximal and distal ends and a lumen therethrough, said mesh structure lumen being configured to receive said first proximal end of said tubular member in said mesh structure lumen,
said proximal end of said mesh structure including a first valve engagement region that is configured to engage a first region of said first proximal end of said tubular member, said distal end of said mesh structure including a second valve engagement region that is configured to engage a second region of said first end of said tubular member,
said mesh structure further including a cardiovascular structure engagement region disposed between said mesh structure proximal and distal ends,
said mesh structure being configured to transition from a pre-deployment configuration with said tubular member disposed in said mesh structure lumen, wherein said mesh structure and tubular member are capable of being disposed at a first cardiovascular valve region, to a post-deployment compressed configuration, wherein said cardiovascular structure engagement region is disposed adjacent cardiovascular tissue disposed proximate said first cardiovascular valve region.

23. The prosthetic valve of claim 22, wherein said first cardiovascular valve region comprises a valve annulus having ventricular and atrial regions, and wherein, when said mesh structure is in said post-deployment configuration, said cardiovascular structure engagement region is anchored to said ventricular and atrial regions.

24. The prosthetic valve of claim 23, wherein said first cardiovascular valve region comprises a mitral valve region.

25. The prosthetic valve of claim 23, wherein said first cardiovascular valve region comprises a tricusped valve region.

26. The prosthetic valve of claim 22, wherein said cardiovascular structure comprises at least one papillary muscle.

27. The prosthetic valve of claim 22, wherein said mesh structure comprises a biocompatible polymeric material.

28. The prosthetic valve of claim 27, wherein said biocompatible polymeric material comprises a polymeric material selected from the group consisting of Dacron® and Artelon®.

29. The prosthetic valve of claim 22, wherein said tubular member comprises a first extracellular matrix (ECM) material.

30. The prosthetic valve of claim 29, wherein said first ECM material is derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.

31. The prosthetic valve of claim 30, wherein said first ECM material further comprises an exogenously added first biologically active agent.

32. The prosthetic valve of claim 31, wherein said first biologically active agent comprises a growth factor selected from the group consisting of a platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-alpha), and placental growth factor (PLGF).

33. The prosthetic valve of claim 31, wherein said first biologically active agent comprises a cell selected from the group consisting of a human embryonic stem cell, fetal cardiomyocyte, myofibroblast, mesenchymal stem cell, autotransplated expanded cardiomyocyte, adipocyte, totipotent cell, pluripotent cell, blood stem cell, myoblast, adult stem cell, bone marrow cell, parenchymal cell, epithelial cell, endothelial cell, mesothelial cell, fibroblast, osteoblast, chondrocyte, exogenous cell, endogenous cell, hematopoietic stem cell, myocardial cell, skeletal cell, multi-potent progenitor cell, unipotent progenitor cell, macrophage, xenogenic cell and allogenic cell.

34. The prosthetic valve of claim 30, wherein said first ECM material further comprises a pharmacological agent selected from the group consisting of antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

35. The prosthetic valve of claim 22, wherein said mesh structure further includes an impermeable liner, said liner being disposed in said mesh structure lumen.

36. The prosthetic valve of claim 35, wherein said liner comprises a first biocompatible material.

37. The prosthetic valve of claim 36, wherein said first biocompatible material comprises a second ECM material, said second ECM material being derived from a mammalian tissue source selected from the group consisting of small intestine submucosa, urinary bladder submucosa, stomach submucosa, central nervous system tissue, epithelium of mesodermal origin, dermal extracellular matrix, subcutaneous extracellular matrix, gastrointestinal extracellular matrix, placental extracellular matrix, ornomentum extracellular matrix, cardiac extracellular matrix, kidney extracellular matrix, pancreas extracellular matrix, lung extracellular matrix, and combinations thereof.
